(19) United States
(12) Patent Application Publication
(10) Pub. No.: US 2016/0041376 A1
(43) Pub. Date: Feb. 11, 2016

(54) SYSTEM AND METHOD FOR MEASURING OIL CONTENT IN WATER USING LASER-INDUCED FLUORESCENT IMAGING

(71) Applicant: Jianfeng Zhang, Sugar Land, TX (US)

(72) Inventor: Jianfeng Zhang, Sugar Land, TX (US)

(73) Assignee: CLEARVIEW SUBSEA LLC, Sugar Land, TX (US)

(21) Appl. No.: 14/455,907

(22) Filed: Aug. 10, 2014

(51) Int. Cl. G02B 21/00; G01B 9/04; G01N 15/02; G01N 33/18; G01N 21/64

(52) U.S. Cl. CPC G02B 21/0076; G01N 33/1833; G01N 21/6402; G01N 15/0227; G02B 21/008; G01B 9/04; G01N 2201/06113; G01N 2201/12

(57) ABSTRACT

The invention is a system and method to measure oil content in water utilizing the fluorescence of oil emitted under excitation by laser. Oil and water mixture is transferred through the system to a measurement section in a microscope, which produces high resolution 3-dimensional images of the oil and water mixture with the fluorescence. The images are analyzed to calculate the amount of oil in water and oil droplets distribution. The image is also analyzed to distinguish oil coated solids from oil droplets, and to calculate the sizes and volumes of the solids.

### FIELD OF THE INVENTION

The field of the invention is devices and methods for the measurement of oil content in water, especially as it relates to the measurement of oil content in water produced from petroleum and natural gas reservoirs.

### BACKGROUND OF THE INVENTION

### Produced Water Discharge Regulation

The global oil industry produces over 300 million barrels of water per day, which amounts to approximately 80% of the liquids produced (Ref. TUV-NEL 2010, Fluor 2012). In offshore production, most of the produced water is discharged into the environment after treatment to discharge standards. A small fraction is re-injected into the formation.

The discharge of produced water is regulated worldwide, with the regulations varying among countries or regions (Fluor, 2012). Oil in water content and toxicity are the two primary regulatory compliance measures. The US regulatory requirements for offshore produced water include oil and grease amount limit (currently 29 mg/L monthly average, 42 mg/L daily max), toxicity test and no free oil discharge, and no sand discharge. The oil and grease measurement is by the EPA 1664A method, which is a gravimetric method using n-hexane solvent extraction and performed in approved laboratories.

### Field Measurements

Field measurements, both bench-top and online, have also been commonly applied for process monitoring and optimization. Prior art on oil content measurement are represented by the following patents:
U.S. Pat. No. 7,935,938 B2, APPARATUS FOR MEASURING FLUORESCENT MATERIAL IN A LIQUID, 2011
U.S. Pat. No. 7,248,363 B2, PARTICLE SIZE ANALYZER, 2007
U.S. Pat. No. 6,525,325 B1, SYSTEM FOR QUANTIFYING THE HYDROCARBON CONTENT OF AQUEOUS MEDIA, 2003
European Patent EP 0 997 732 A1, METHOD AND EQUIPMENT FOR CONTINUOUS MEASUREMENT OF MINERAL OILS IN WATER BY MEANS OF SPECTROPHOTOMETRIC DETECTOR, 1999
U.S. Pat. No. 5,381,002, FLUORESCENCE METHOD OF QUANTIFYING HYDROCARBONS, INCLUDING CRUDE OIL, DISPERSED IN WATER, 1993
U.S. Pat. No. 4,953,978, PARTICLE SIZE ANALYSIS UTILIZING POLARIZATION INTENSITY DIFFERENTIAL SCATTERING, 1990

In prior art, laser induced fluorescence has only been used for emission magnitude measurement. Imaging analysis has only used with on images obtained by direct lighting and reflection.

In imaging analysis method, the presently available systems for produced water can resolve particles 2-3 microns in size or larger (Tyrie et al. 2005).

The offshore produced water treatment system typically removes oil droplets 3-5 microns and larger, based on the performance of gas induced flotation units as reported by Argonne National Laboratory (Hayes et al. 2004). Finborud et al (1999) discussed that the oil droplet sizes downstream of the first stage separator, and commented that field experience had shown that the oil droplet size distribution often has a peak around 10-15 microns. It can then be estimated from the data provided by Finborud et al (1999) that, if oil droplets larger than 3-5 microns are removed from the produced water by the last stage treatment (for example induced gas flotation with chemical injection), 40% or more of the remaining droplets are smaller than 2 microns.

Therefore an imaging analysis method which can resolve the oil droplets smaller then 2 microns in diameter can improve the accuracy of measurements. Contemplated invention described below is such a system and method.

### SUMMARY OF THE INVENTION

Contemplated invention is an oil in water content measurement system and method which utilize laser fluorescence microscopy to generate one or more images, and analyze the images to determine the oil droplet content by the number and size distribution of the droplets. The images can also detect oil coated sand particles. The images can be 3-dimensional for configurations intended for high measurement accuracy, and 2-dimensional for configurations where lower accuracy but higher speed of measurement is desired.

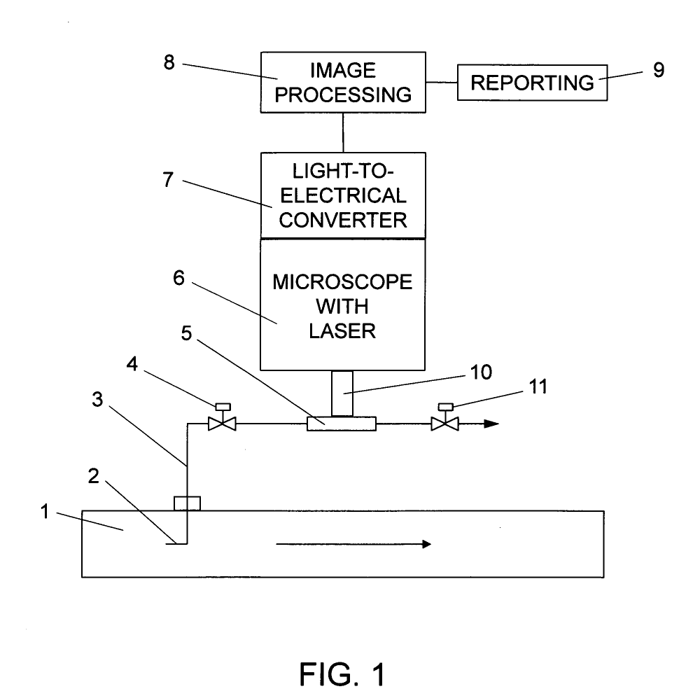

Referring to FIG. 1, a measurement section 5, which is along side the produced water discharge pipe 1, is filled with a sample by opening and closing two valves (4 and 11) on the sampling path 3. A microscope 6 uses laser to scan multiple view volumes of the sample. The fluorescent emissions produced by the sample are recorded by light-to-electrical signal converter and recoder 7 to form a 2-dimensional (2-D) image of the focal plane which at a certain location in the sample from the microscope's objective 10. A particularly preferred configuration is laser fluorescence confocal microscope with spinning disks. Other microscopes for generating fluorescence with laser are also possible. Charge-coupled device (CCD) is particularly preferred for the light to electrical signal converter, but converters with the necessary sensitivity are also possible.

The stacks of 2-D images are processed by an image processing computing device 8 which can be either at the measurement site or at a remote location. A particularly preferred image processing algorithm is deconvolution to form 3-dimensional (3-D) images of the view volumes. The 3-D images are analyzed to determine the oil droplets' volume content and size distribution. In other applications, the 2-dimensional images are directly used to identify the oil droplets and solid particles.

### DETAILED DESCRIPTION

The inventor has discovered that the fluorescent properties of certain components of oil, such as the Polycyclic aromatic hydrocarbons which emits fluorescent light when experienced excitation by laser light, can be used to produce an image with a microscope. The inventor also discovered that the image can have very high resolution (250 nanometer or even finer), and can be analyzed with an image analysis algorithm to determine the oil droplets' number, size distribution and volume. The inventor further discovered that the method can be used to measure the number, sizes, shape and volume of oil coated solid particles.

The measurement setup of the new method is illustrated in FIG. 1, where:
1 is a conduit carrying produced water to discharge, re-injection or to other locations
2 is a produced water sampling device, such as a tube inserted at the appropriate location within the produced water conduit 1
3 is the piping to fluidly couple the sampling device with a valve 4
4 is a valve which can be opened or closed to allow the sampled fluid from 3 to enter measurement section 5 or isolate the sample in measurement section 5
5 is the measurement section where the sample resides and is scanned by the microscope 6
6 is a microscope which includes a laser source. A particularly preferred microscope is laser fluorescence confocal microscope.
7 is a device converting the fluorescent emission signal to electrical signal. A particularly preferred camera is charge-coupled device (CCD).
8 is an image processing unit. A particularly preferred configuration is a computer at the measurement site. Another particularly preferred configuration is a computing system embedded in the microscope body or camera body. Another preferred configuration is a computer at a remote site, which is connected with the camera with signal linkage. The image processing unit uses one or more algorithms to process the image, such as with the particularly preferred method of deconvolution, and identify the objects in the image as oil droplets, solid particles, or other.
9 is a reporting device for the user to obtain the determined oil droplets and other particles' sizes, shapes and volumes.
10 is the objective of the microscope. The objective can be positioned outside the view section, in which case the measurement section has a viewing window, or can be positioned inside the measurement section.
11 is a valve which works together with valve 4 (opened or closed) to allow the sampled fluid from 3 to enter measurement section 5 or isolate the sample in measurement section 5.

A water sampling device 2 is inserted to the produced water discharge pipe 1 for a slip stream to be flown through the sample piping 3, valves 4 and 11, measurement section 5 and to discharge. The measurement section is instrumented with a microscope 6 with laser, such as the particularly preferred spinning disk laser confocal microscope with a scanning unit unit with lenslet. The microscope illuminates the view volume with laser beam in a scanning manner. The fluorescence generated by the oil droplets in the sample is captured by the light to electrical signal converter 7, a particularly preferred configuration for which is a CCD (charge-coupled device). The digital signals from the converter are sent to an image processing computing device 8 which can be located either at the site, at a remote location. The image processing utilizes algorithms to improve the image quality if needed, for example using the particularly preferred algorithms of deconvolution, to remove the noise caused by light contributions from out-of-focal plane locations. The processed signals are analyzed to determine the location and size of the oil droplets in the sample. The total volume fraction of the oil droplets and the size distribution are reported through the human machine interface 9.

Many measurement sequences can be utilized with the contemplated invention. An example measurement sequence is as follows:
1. The valves in the sampling flow path are opened to initiate the measurement operation.
2. After a period of pre-determined time, the flow in the measurement section reaches equilibrium, and the valves are closed.
3. Wait until water motion ceases. At this time the scanning of the sample can begin.
4. One view volume is chosen for scanning. The laser beams excite the aromatic hydrocarbon molecules in the oil droplets to generate fluorescent emissions. The fluorescent emissions are captured by the CCD. Once one focal plane has been scanned (one frame), the adjacent focal plane is selected. This repeats until the sample volume has been completely scanned.
5. Another view volume is chosen and scanned. This repeats until all the view volumes are scanned. It takes several seconds to complete the scan of one view volume.
6. The captured signals are processed by the image processing unit for oil content and particle size distribution readout.
7. The valves are opened to discharge the sample.
8. The measurement unit is ready for the next measurement.

Many variations of the measurement configuration and image processing method are possible, including:
The measurement section is not fluidly coupled with the sampling path, instead, it is a separate sample contained in a transparent device under the objective, similar to a typical microscope configuration. Thus, the method can be utilized in a laboratory on separately collected and prepared samples.
The microscope is a wide-field laser fluorescence microscopy, a multiphoton microscope.
The image analysis unit uses the stack of 2-D images from the light to electrical signal converter directly, without first performing noise reduction, for object identification
The image analysis unit uses deconvolution on the images collected by the wide field microscope
Only the 2-D image of a single focal plane is taken at each location for increasing the scanning speed.

The above has disclosed the specifics of the contemplated invention to measure oil content in water. It should be apparent to those skilled in the art that many other variations and modifications are possible which are within the spirit of the disclosed invention.

1. A measurement system for determining the amount of oil in water and the distribution of oil droplet sizes, comprising
a. Water sampling piping and valves;
b. A measurement section between the valves;
c. A microscope with a laser source, which illuminates the sample in the measurement section with laser;
d. A light to electrical signal converter for converting the laser induced fluorescent;
emissions from the sample in measurement section to images;
e. An image processing unit;
f. A reporting device which is a human-machine interface.

2. The measurement system in claim 1 wherein the measurement section has a view window for the microscope or within the measurement section;

3. The measurement system in claim 1 wherein the microscope utilizes laser to illuminate the a portion or the whole of the sample in the measurement section at any time instant during the measurement;

4. The measurement system in claim 1 wherein the microscope is a confocal microscope with laser, a multi-photon microscope with laser or a wide field microscope with laser;

5. The measurement system in claim 1 wherein the light to electrical signal converter is a charge-coupled device or other converter;

6. The measurement system in claim 1 wherein the light to electrical signal converter converts the light signal into electrical signal to form an image or images of the sample;

7. The measurement system in claim 1 wherein the imaging processing unit is a computing device at the site of measurement;

8. The measurement system in claim 1 wherein the imaging processing unit is a computing device at a different location from the site of measurement, and is connected with the camera with signal linkage;

9. The measurement system in claim 1 wherein the imaging processing unit is a computing device integrated into the microscope;

10. The measurement system in claim 1 wherein the measurement section is not connected with sampling piping, and the measurement section contains a sample that is separately obtained.

11. A method of measuring the amount of oil in water and the distribution of oil droplet sizes, comprising:
a. The valves in the sampling flow path are opened to initiate the measurement operation. The valves are closed after equilibrium of flow has been established, so that the sample is isolated in the measurement section;
b. The microscope illuminates the sample and record the image. The illumination can be in a scanning manner or across the whole view volume at the same instant;
c. In the scanning method, the laser systematically scans through the points in the view volumes. The fluorescent emissions are captured by the light to electrical signal converter for form images;
d. The captured signals are processed by the image processing unit for oil content and particle size distribution readout.

12. The method of claim 11 wherein the microscope scans one focal plane for a 2-dimensional image, which is analyzed for oil content, including the total oil volume fraction in water and the droplet size distribution;

13. The method of claim 11 wherein the microscope scans multiple focal planes for form a stack of images, which are used to generate a 3-dimensional image;

14. The method of claim 11 wherein the 3-dimensional image of claim 13 is analyzed for oil content, including the total oil volume fraction in water and the droplet size distribution;

15. The method of claim 11 wherein the 3-dimensional image of claim 13 is generated by deconvolution of 2-dimensional images;

16. The method of claim 11 wherein the 3-dimensional image generation is by combination of the 2-dimensional images from the confocal microscope or multi-photon microscope of claim 4;

17. The method of claim 11 wherein the 3-dimensional image generation is by deconvolution of the 2-dimensional images from the confocal microscope or multi-photon microscope of claim 4;

18. The method of claim 11 wherein the image or images are analyzed for measuring solid particle size distribution, shapes, and total volume fraction in water.
